(19) United States
(12) Patent Application Publication
(10) Pub. No.: US 2026/0115407 A1
(43) Pub. Date: Apr. 30, 2026

(54) SENSORY STIMULATION KIT

(71) Applicant: Whitney Vik, Little Falls, NY (US)

(72) Inventor: Whitney Vik, Little Falls, NY (US)

(21) Appl. No.: 18/931,789

(22) Filed: Oct. 30, 2024

(51) Int. Cl. A61M 21/00

(52) U.S. Cl. CPC A61M 21/00; A61M 2021/0022; A61M 2021/0027; A61M 2021/0044; A61M 2205/0216; A61M 2205/0233; A61M 2205/10; A61M 2205/3327; A61M 2205/3331; A61M 2205/50; A61M 2205/581

(57) ABSTRACT

A sensory stimulation kit for facilitating coping behaviors for users with sensory processing difficulties includes a plurality of sensory modules which are unattached to each other. Each sensory module is configured to provide a stimulus. The plurality of sensory modules includes a lighting module which comprises a body, a plurality of light sources, and a lighting control interface. The plurality of light sources is mounted on the body and is configured to emit light in a selected one of a plurality of lighting patterns. The lighting control interface is operatively coupled to the plurality of light sources for selecting one of the plurality of lighting patterns.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to self-stimulating devices and more particularly pertains to a new self-stimulating device for facilitating coping behaviors for users with sensory processing difficulties.

### (2) Description of Related Art Including Information Disclosed Under 37 CFR 1.97 and 1.98

Individuals with autism, attention deficit hyperactivity disorder, or similar conditions may face challenges in processing sensory stimuli and may engage in self-stimulatory behaviors or “stimming” as a means of coping with overstimulation and anxiety. Such self-stimulatory behaviors include making repetitive physical movements, moving objects, making sounds, and other behaviors and have been known to enable individuals to cope with overstimulation or other sources of distress.

The prior art includes various sensory stimulating apparatuses which facilitate various forms of self-stimulatory behaviors. However, the prior art fails to describe a kit in which a plurality of disconnected modules provide different stimuli and may be held and displayed via a transparent container. Such a kit allows individuals to select one or more modules from the plurality for use by looking through the transparent container to the modules held inside and enable the individual to engage in self-stimulatory behavior with the selected module(s) independently of the other modules.

### BRIEF SUMMARY OF THE INVENTION

An embodiment of the disclosure meets the needs presented above by generally comprising a plurality of sensory modules which are unattached to each other. Each sensory module is configured to provide a stimulus. The plurality of sensory modules includes a lighting module which comprises a body, a plurality of light sources, and a lighting control interface. The plurality of light sources is mounted on the body and is configured to emit light in a selected one of a plurality of lighting patterns. The lighting control interface is operatively coupled to the plurality of light sources for selecting one of the plurality of lighting patterns.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a sensory stimulation kit according to an embodiment of the disclosure.

FIG. 2 is a kit view of a plurality of sensory modules of an embodiment of the disclosure.

FIG. 3 is a kit view of a plurality of figure modules of an embodiment of the disclosure.

FIG. 4 is a rear view of a container of an embodiment of the disclosure.

FIG. 5 is a side view of a container of an embodiment of the disclosure.

FIG. 6 is a perspective view of a container of an embodiment of the disclosure.

### DETAILED DESCRIPTION OF THE INVENTION

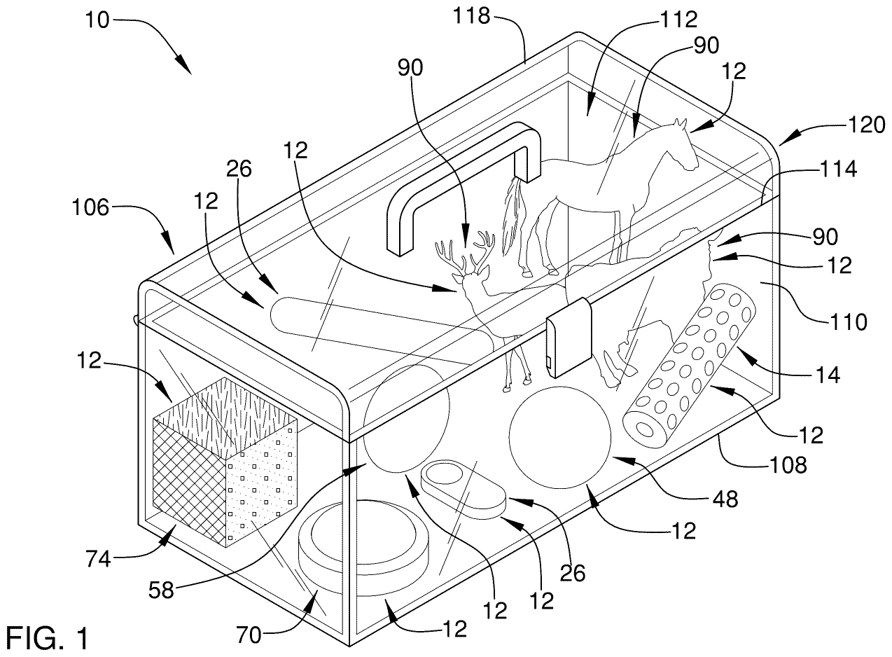

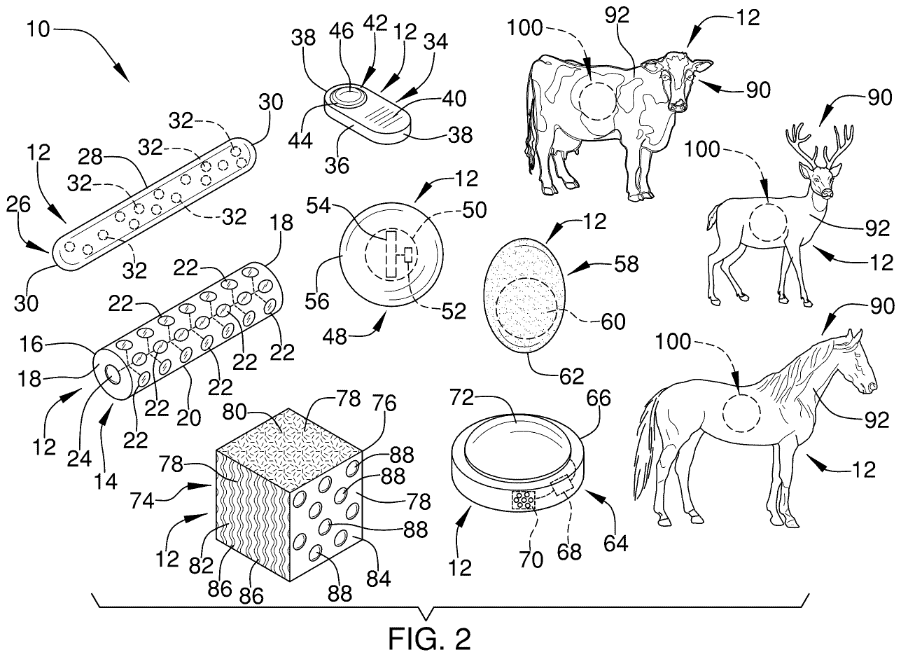

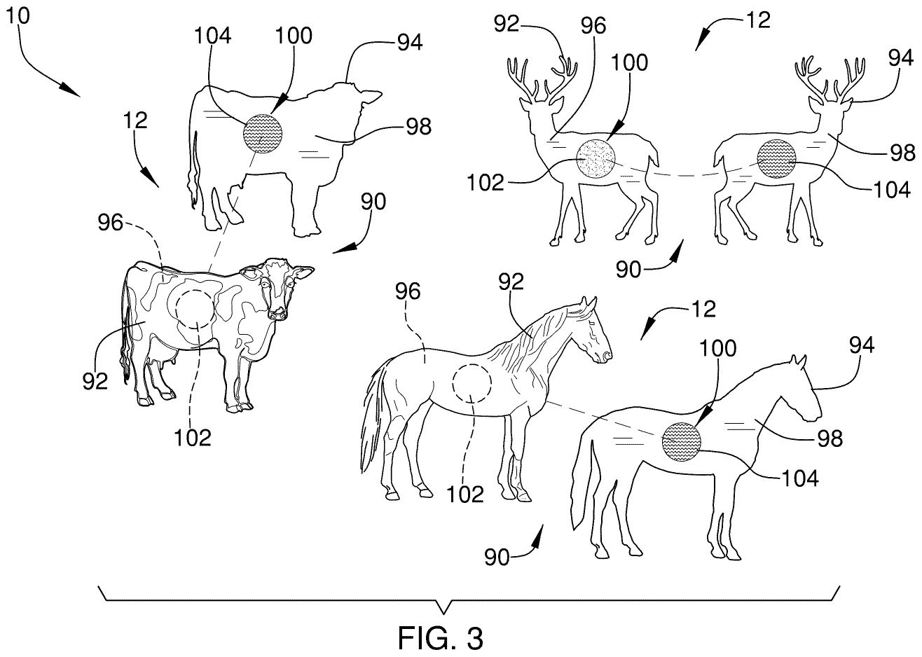

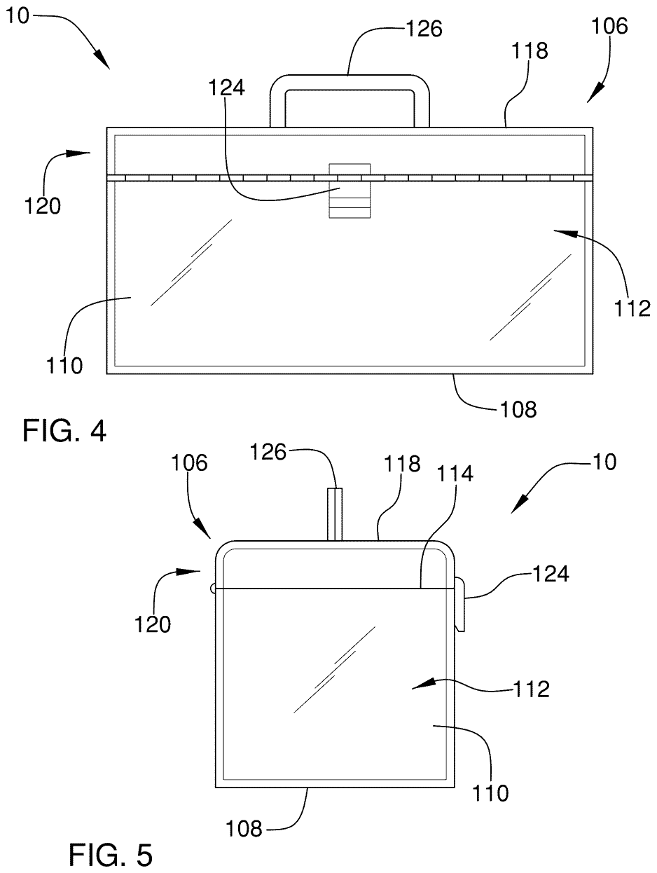

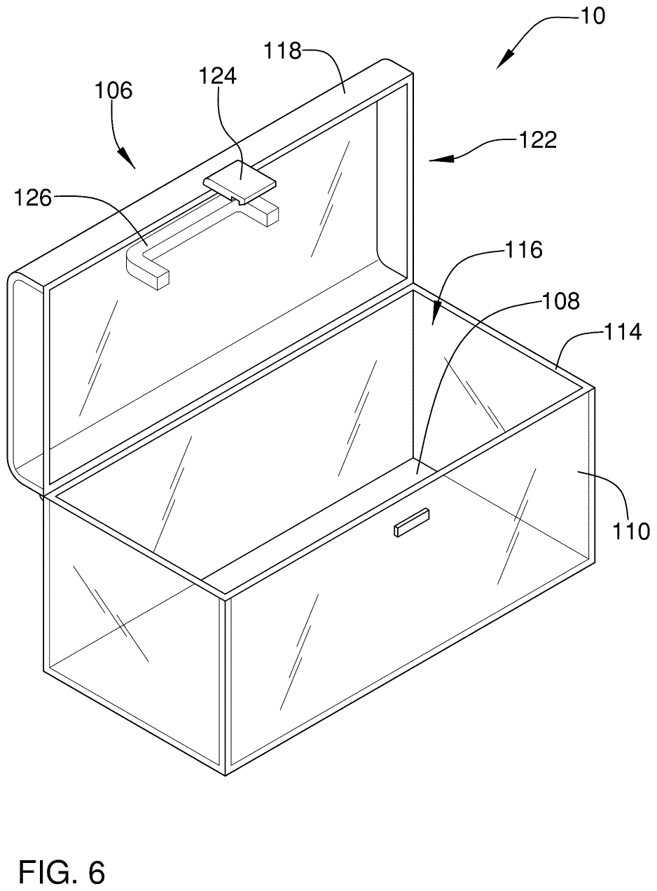

With reference now to the drawings, and in particular to FIGS. 1 through 6 thereof, a new self-stimulating device embodying the principles and concepts of an embodiment of the disclosure and generally designated by the reference numeral 10 will be described.

As best illustrated in FIGS. 1 through 6, the sensory stimulation kit 10 generally comprises a plurality of sensory modules 12 that are unattached to each other. Each sensory module 12 of the plurality of sensor modules is configured to provide a stimulus which may have therapeutic benefit for a user with autism, attention deficit hyperactivity disorder, or similar conditions. The sensory modules 12 facilitate the user in engaging in self-stimulatory behavior or “stimming” which may help the user calm themselves or otherwise cope with various stresses.

The plurality of sensory modules 12 includes a lighting module 14 which comprises a body 16 that is elongated between a pair of ends 18. The body 16 has a peripheral surface 20 which extends between the pair of ends 18 and which has a cylindrical shape. The body 16 may have a soft material such as nylon, polyester, silk, silicone, rubber, or the like. A plurality of light sources 22 is mounted on the body 16. The light sources 22 are distributed across the peripheral surface 20 of the body 16 and are configured to emit light in a selected one of a plurality of lighting patterns. The light sources 22 are evenly spaced between the pair of ends 18 of the body 16 and are evenly spaced radially around the peripheral surface 20. A lighting control interface 24 is operatively coupled to the plurality of light sources 22 for selecting one of the lighting patterns. The lighting control interface 24 comprises a button which is positioned at one of the pair of ends 18 of the body 16. The button may be pressed to toggle through the lighting patterns. In other embodiments, the lighting control interface 24 may comprise a switch, a dial, a touchscreen, or any other suitable input means.

Another one of the sensory modules 12 is a shaker module 26 which comprises a tube 28 and a plurality of particulates 32 housed loosely in the tube 28. The tube 28 has closed ends 30, and the particulates 32 are configured to produce an audible sound when striking the tube 28. The materials of the tube 28 and the particulates 32 may, for example, be selected for their ability to resonate to produce an audible sound rather over materials with sound-dampening properties. The user may shake or tilt the shaker module 26 to cause the particulates 32 to be launched or fall within the tube 28, thereby striking the tube 28 and causing the audible sound.

The plurality of sensory modules 12 further includes a clicker module 34 which comprises a base plate 36 and a clicking button 42. The base plate 36 is elongated between a pair of ends 38 and has a top side 40 which extends between the pair of ends 38 of the base plate 36. The clicking button 42 is mounted on the top side 40 of the base plate 36 via an outer edge 44 of the clicking button 42. The clicking button 42 comprises a resiliently bendable material such that, when a center portion 46 of the clicking button 42 is depressed from a rest position and released, the clicking button 42 returns to the rest position. The clicking button 42 is configured to emit an audible clicking or popping sound when resiliently returning to the rest position. Other clicking mechanisms may be used which produce a clicking sound when depressed or released. The resiliently bendable material comprises a sheet metal but may comprise a plastic material or other suitable material. The clicking button 42 is positioned adjacent to one end 38 of the pair of ends 38 of the base plate 36 and is spaced from another end 38 of the pair of ends 38 of the base plate 36. The user may depress and release the clicking button 42 to hear the clicking sound and to feel the tactile reverberations which may transfer through the user's hand.

The plurality of sensory modules 12 also includes a vibrator module 48 which comprises a core housing 50 that is coupled to and surrounded by an outer layer 56. A vibration motor 52 is mounted in the core housing 50. A pressure sensor 54 is mounted in the core housing 50 and is operatively coupled to the vibration motor 52. The pressure sensor 54 is configured to detect pressure exerted on the core housing 50 and activate the vibration motor 52 when detecting a pressure input exerted on the housing. The outer layer 56 comprises a resiliently compressible material such as a rubber, a silicone, a plastic foam, a rubber foam, or the like. When the user squeezes the outer layer 56, the outer layer 56 compresses and transfers the input squeezing force to the pressure sensor 54, which thereby activates the vibration motor 52 to cause the vibrator module 48 to vibrate in the user's hand.

Another sensory module 12 includes a squeezable module 58 which comprises an inner member 60 and an outer member 62. The inner member 60 comprises a rigid material and has a spherical shape. The outer member 62 is coupled to and surrounds the inner member 60. The outer member 62 comprises a resiliently deformable material and has an ovoid shape. The rigid material may comprise, for example, a ceramic or other mineral, a metal, or a sufficiently rigid plastic. The resiliently deformable material may comprise a rubber, a silicone, a plastic foam, a rubber foam, or the like. The user may squeeze the outer member 62 to feel the outer member 62 deform until the user squeezes hard enough to press against the inner member 60 which does not significantly yield to the user's grip.

The plurality of sensory modules 12 also includes a sound playing module 64 which comprises a base housing 66, a processor 68, a sound emitter 70, and a sound control input 72. The processor 68 is mounted in the base housing 66 and has a memory which is used to store a plurality of audio files. The sound emitter 70 is mounted in the base housing 66 and is operably coupled to the processor 68. The sound control input 72 is mounted atop the base housing 66 and is operable to select from the plurality of audio files stored on the memory of the processor 68 to be played via the sound emitter 70. The sound control input 72 comprises a button which may toggle through the plurality of audio files but may include any suitable input means such as a dial, a switch, a touch screen, or the like. Audio files may include data for audibly playing soothing sounds such as a ticking sound, nature sounds, a heartbeat, repetitive noises, or the like. The base housing 66 has a disc shape or hockey-puck shape and may use a soft material such as nylon, polyester, silk, silicone, rubber, or the like. The user may select one of the audio files as desired.

Another sensory module 12 is a texture module 74 which comprises a cube 76 or other polyhedron with a plurality of faces 78. Each face 78 of the plurality of faces 78 has an associated texture. The plurality of faces 78 includes a first face 80 which comprises a fabric that has a pile weave. The pile weave of the fabric of the first face 80 may have a generally even pile and may includes a soft material such as fur, faux fur, polyester, nylon, acrylic, or the like. A second face 82 of the plurality of faces 78 defines a plurality of ribs 86 which are oriented parallel to each other and have sinusoidal shapes. A third face 84 of the plurality of faces 78 defines a plurality of protrusions 88 with circular shapes which are spaced from each other and distributed across the third face 84. The materials on the second face 82 and the third face 84 may also have a fabric with a pile weave, but whose pile is uneven to define the ribs 86 and protrusions 88 respectively. Various other textures of differing geometries may be used for other faces 78 of the plurality of faces 78. The user may feel the various textures of the different faces 78 as desired to self-stimulate.

The plurality of sensory modules 12 further includes a plurality of figurine modules 90. Each figurine module 90 comprises a first segment 92 and a second segment 94 that removably coupled together. A first mating surface 96 of the first segment 92 is coextensive with a second mating surface 98 of the second segment 94. A connector 100 couples the first segment 92 to the second segment 94. The connector 100 comprises a first mating member 102 which is coupled to the first segment 92 and a second mating member 104 which is coupled to the second segment 94. The connector 100 comprises a hook-and-loop fastener but may comprise a snap button fastener, a magnetic fastener, or the like. The figurine modules 90 may be shaped to depict animals or other three-dimensional shapes. The user may attach and detach the first segment 92 and the second segment 94 of each figurine module 90.

A container 106 is provided for holding and displaying the plurality of sensory modules 12. The container 106 comprises a bottom wall 108 and a perimeter wall 110, wherein the perimeter wall 110 is coupled to and extends upwardly from the bottom wall 108. The bottom wall 108 and the perimeter wall 110 define an interior space 112 of the container 106, and a top edge 114 of the perimeter wall 110 defines an opening 116 to the interior space 112. The container 106 further comprises a lid 118 coupled to the perimeter wall 110 which is pivotable between a closed position 120 in which the lid 118 covers the opening 116 to the interior space 112 and an open position 122 in which the opening 116 is uncovered by the lid 118. The bottom wall 108, the perimeter wall 110, and the lid 118 comprise a transparent material such that the interior space 112 is visible through the bottom wall 108, the perimeter wall 110, and the lid 118. The container 106 further comprises a closure 124 coupled to the lid 118 which is operable to secure the lid 118 in the closed position 120. The closure 124 comprises a latch but may comprise a snap fit fastener, a magnetic fastener, a hook-and-loop fastener, or the like. The container 106 further comprises a handle 126 which is coupled to a top side of the lid 118.

In use, the user may use the various sensory modules 12 as described above. The user may identify and select one of the sensory modules 12 by viewing them through the transparent material of the container 106 while the lid 118 of the container 106 is secured in the closed position 120 and holds the sensory modules 12 therein. The handle 126 may be used to carry the container 106 and to open the lid 118.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A sensory stimulation kit for facilitating therapeutic self-stimulatory behavior, the sensory stimulation kit comprising:
a plurality of sensory modules, the plurality of sensory modules being unattached to each other, each sensory module of the plurality of sensor modules being configured to provide a stimulus, the plurality of sensory modules including:
a lighting module comprising:
a body;
a plurality of light sources mounted on the body, the plurality of light sources being configured to emit light in a selected one of a plurality of lighting patterns; and
a lighting control interface operatively coupled to the plurality of light sources for selecting one of the plurality of lighting patterns.

2. The sensory stimulation kit of claim 1, wherein:
the body of the lighting module is elongated between a pair of ends, the body having a peripheral surface which extends between the pair of ends;
the plurality of light sources of the lighting module is distributed across the peripheral surface of the body; and
the lighting control interface of the lighting module is positioned at one of the pair of ends of the body.

3. The sensory stimulation kit of claim 2, wherein:
the peripheral surface of the body of the lighting module has a cylindrical shape; and
the plurality of light sources of the lighting module is evenly spaced between the pair of ends of the body, the plurality of light sources being evenly spaced radially around the peripheral surface.

4. The sensory stimulation kit of claim 1, wherein the plurality of sensory modules further includes a shaker module comprising:
a tube with a pair of closed ends; and
a plurality of particulates housed loosely in the tube, the plurality of particulates being configured to produce an audible sound when striking the tube.

5. The sensory stimulation kit of claim 1, wherein the plurality of sensory modules further includes a clicker module comprising:
a base plate, the base plate being elongated between a pair of ends, the base plate having a top side which extends between the pair of ends of the base plate; and
a clicking button mounted on the top side of the base plate via an outer edge of the clicking button, the clicking button comprising a resiliently bendable material such that, when a center portion of the clicking button is depressed from a rest position and released, the clicking button returns to the rest position, the clicking button being configured to emit an audible clicking sound when resiliently returning to the rest position.

6. The sensory stimulation kit of claim 5, wherein the clicking button of the clicker module is positioned adjacent to one end of the pair of ends of the base plate and being spaced from another end of the pair of ends of the base plate.

7. The sensory stimulation kit of claim 5, wherein the resiliently bendable material of the clicking button of the clicker module comprises a sheet metal.

8. The sensory stimulation kit of claim 1, wherein the plurality of sensory modules further includes a vibrator module comprising:
a core housing;
a vibration motor mounted in the core housing;
a pressure sensor mounted in the core housing and operatively coupled to the vibration motor, the pressure sensor being configured to detect pressure exerted on the core housing, the pressure sensor being configured to activate the vibration motor when the pressure sensor detects a pressure input exerted on the housing; and
an outer layer coupled to and surrounding the core housing, the outer layer comprising a resiliently compressible material.

9. The sensory stimulation kit of claim 1, wherein the plurality of sensory modules further includes a squeezable module comprising:
an inner member comprising a rigid material; and
an outer member coupled to and surrounding the inner member, the outer member comprising a resiliently deformable material.

10. The sensory stimulation kit of claim 9, wherein:
the inner member of the squeezable module has a spherical shape; and
the outer member of the squeezable module has an ovoid shape.

11. The sensory stimulation kit of claim 1, wherein the plurality of sensory modules further includes a sound playing module comprising:
a base housing;
a processor mounted in the base housing, the processor having a memory;
a sound emitter mounted in the base housing and operably coupled to the processor; and
a sound control input operatively coupled to the processor, the sound control input being operable to select from a plurality of audio files stored on the memory of the processor to be played via the sound emitter.

12. The sensory stimulation kit of claim 1, wherein the plurality of sensory modules further includes a texture module comprising a polyhedron with a plurality of faces, each face of the plurality of faces having an associated texture, the associated texture of each face being tactilely distinct from the associated texture of at least one of the other faces of the plurality of faces.

13. The sensory stimulation kit of claim 12, wherein the polyhedron of the texture module is a cube.

14. The sensory stimulation kit of claim 12, wherein the plurality of faces of the polyhedron of the texture module includes:
a first face comprising a fabric having a pile weave;
a second face defining a plurality of ribs, the plurality of ribs being oriented parallel to each other, each rib of the plurality of ribs having a sinusoidal shape; and
a third face defining a plurality of protrusions, the plurality of protrusions being spaced from each other, each protrusion of the plurality of protrusions having a circular shape.

15. The sensory stimulation kit of claim 1, wherein the plurality of sensory modules further includes a figurine module comprising:
a first segment and a second segment removably coupled together, a first mating surface of the first segment being coextensive with a second mating surface of the second segment; and
a connector coupling the first segment to the second segment, the connector comprising a first mating member coupled to the first segment and a second mating member coupled to the second segment.

16. The sensory stimulation kit of claim 15, wherein the connector comprises a hook-and-loop fastener.

17. The sensory stimulation kit of claim 1, further comprising a container for holding the plurality of sensory modules, the container comprising a bottom wall and a perimeter wall, the perimeter wall being coupled to and extending upwardly from the bottom wall, the bottom wall and the perimeter wall defining an interior space of the container, a top edge of the perimeter wall defining an opening to the interior space, the container further comprising a lid coupled to the perimeter wall, the lid being pivotable between a closed position in which the lid covers the opening to the interior space and an open position in which the opening is uncovered by the lid, wherein the bottom wall, the perimeter wall, and the lid comprise a transparent material such that the interior space is visible through the bottom wall, the perimeter wall, and the lid, the container further comprising a closure coupled to the lid, the closure being operable to secure the lid in the closed position, the closure comprising a latch, the container further comprising a handle coupled to a top side of the lid.

18. A sensory stimulation kit for facilitating therapeutic self-stimulatory behavior, the sensory stimulation kit comprising:
a plurality of sensory modules, the plurality of sensory modules being unattached to each other, each sensory module of the plurality of sensor modules being configured to provide a stimulus, the plurality of sensory modules including:
a lighting module comprising:
a body, the body being elongated between a pair of ends, the body having a peripheral surface which extends between the pair of ends, the peripheral surface having a cylindrical shape;
a plurality of light sources mounted on the body, the plurality of light sources being distributed across the peripheral surface of the body, the plurality of light sources being configured to emit light in a selected one of a plurality of lighting patterns, the plurality of light sources being evenly spaced between the pair of ends of the body, the plurality of light sources being evenly spaced radially around the peripheral surface; and
a lighting control interface operatively coupled to the plurality of light sources for selecting one of the plurality of lighting patterns, the lighting control interface being positioned at one of the pair of ends of the body;
a shaker module comprising:
a tube with a pair of closed ends; and
a plurality of particulates housed loosely in the tube, the plurality of particulates being configured to produce an audible sound when striking the tube;
a clicker module comprising:
a base plate, the base plate being elongated between a pair of ends, the base plate having a top side which extends between the pair of ends of the base plate; and
a clicking button mounted on the top side of the base plate via an outer edge of the clicking button, the clicking button being positioned adjacent to one end of the pair of ends of the base plate and being spaced from another end of the pair of ends of the base plate, the clicking button comprising a resiliently bendable material such that, when a center portion of the clicking button is depressed from a rest position and released, the clicking button returns to the rest position, the clicking button being configured to emit an audible clicking sound when resiliently returning to the rest position, the resiliently bendable material comprising a sheet metal;
a vibrator module comprising:
a core housing;
a vibration motor mounted in the core housing;
a pressure sensor mounted in the core housing and operatively coupled to the vibration motor, the pressure sensor being configured to detect pressure exerted on the core housing, the pressure sensor being configured to activate the vibration motor when the pressure sensor detects a pressure input exerted on the housing; and
an outer layer coupled to and surrounding the core housing, the outer layer comprising a resiliently compressible material;
a squeezable module comprising:
an inner member comprising a rigid material, the inner member having a spherical shape; and
an outer member coupled to and surrounding the inner member, the outer member comprising a resiliently deformable material, the outer member having an ovoid shape;
a sound playing module comprising:
a base housing;
a processor mounted in the base housing, the processor having a memory;
a sound emitter mounted in the base housing and operably coupled to the processor; and
a sound control input operatively coupled to the processor, the sound control input being operable to select from a plurality of audio files stored on the memory of the processor to be played via the sound emitter;
a texture module comprising a polyhedron with a plurality of faces, each face of the plurality of faces having an associated texture, the polyhedron being a cube, the plurality of faces including:
a first face comprising a fabric having a pile weave;
a second face defining a plurality of ribs, the plurality of ribs being oriented parallel to each other, each rib of the plurality of ribs having a sinusoidal shape; and
a third face defining a plurality of protrusions, the plurality of protrusions being spaced from each other, each protrusion of the plurality of protrusions having a circular shape; and
a plurality of figurine modules, each figurine module of the plurality of figurine modules comprising:
a first segment and a second segment removably coupled together, a first mating surface of the first segment being coextensive with a second mating surface of the second segment; and
a connector coupling the first segment to the second segment, the connector comprising a first mating member coupled to the first segment and a second mating member coupled to the second segment, the connector comprising a hook-and-loop fastener; and
a container for holding the plurality of sensory modules, the container comprising a bottom wall and a perimeter wall, the perimeter wall being coupled to and extending upwardly from the bottom wall, the bottom wall and the perimeter wall defining an interior space of the container, a top edge of the perimeter wall defining an opening to the interior space, the container further comprising a lid coupled to the perimeter wall, the lid being pivotable between a closed position in which the lid covers the opening to the interior space and an open position in which the opening is uncovered by the lid, wherein the bottom wall, the perimeter wall, and the lid comprise a transparent material such that the interior space is visible through the bottom wall, the perimeter wall, and the lid, the container further comprising a closure coupled to the lid, the closure being operable to secure the lid in the closed position, the closure comprising a latch, the container further comprising a handle coupled to a top side of the lid.
